(19) United States
(12) Patent Application Publication
(10) Pub. No.: US 2014/0336692 A1
(43) Pub. Date: Nov. 13, 2014

(54) RUPTURE-RESISTANT COMPLIANT RADIOPAQUE CATHETER BALLOON AND METHODS FOR USE OF SAME IN AN INTRAVASCULAR SURGICAL PROCEDURE

(71) Applicant: R4 VASCULAR, INC., Maple Grove, MN (US)

(72) Inventors: Steven J. Allex, Shoreview, MN (US); Donald Geer, Plymouth, MN (US); Brett Allyn Williams, Lino Lakes, MN (US); Brady Jon Hatcher, Rogers, MN (US); Tristan Lynn Tieso, Fridley, MN (US)

(21) Appl. No.: 14/340,407

(22) Filed: Jul. 24, 2014

Related U.S. Application Data
(63) Continuation of application No. 12/610,102, filed on Oct. 30, 2009.
(60) Provisional application No. 61/109,840, filed on Oct. 30, 2008.

(51) Int. Cl. A61M 25/10

(52) U.S. Cl. CPC A61M 25/104; A61M 2025/1075; A61M 2025/1079
USPC 606/194

(57) ABSTRACT

The present invention provides a compliant balloon for use with a catheter having an inner compliant inner layer defining a cylindrical lumen encased by a fiber layer including non-braided inelastic fibers imparting integrity to the balloon wall. The balloon further includes radiopaque material which may be disposed over substantially the entire length of the balloon as a coating or by incorporation within the fiber layer or an outer coating layer. The balloon is expandable from a folded deflated state to an inflated state by increasing pressure within the balloon and can be used with saline as the sole inflation medium to allow rapid deflation as compared to use of a balloon with a contrast medium.

### CROSS REFERENCE TO RELATED APPLICATION(S)

This application is a continuation application of U.S. application Ser. No. 12/610,102, filed Oct. 30, 2009, now pending; which claims the benefit of priority under 35 U.S.C. §119(e) of U.S. Ser. No. 61/109,840, filed Oct. 30, 2008, the entire content of which is incorporated herein by reference.

### BACKGROUND OF THE INVENTION

1. Field of the Invention

The invention relates generally to medical devices and more specifically to radiopaque catheter balloons for use with balloon catheters.

2. Background Information

Balloon catheters are used in various medical procedures to treat lesions in intraluminal body cavities, predominantly within vascular vessels and arteries, as well as the urethra. Accurate placement of the balloon with respect to the portion of the body vessel being treated is critical, as misplacement can reduce therapeutic efficacy and potentially cause harm to the patient.

One widely used procedure that illustrates how balloon catheters are typically employed is percutaneous transluminal coronary angioplasty (PTCA) for treatment of heart disease. In a typical PTCA procedure, a dilation balloon catheter is advanced over a guidewire to a desired location within the patient's coronary anatomy to position the balloon of the dilation catheter within the stenosis to be dilated.

In an effort to improve accurate placement of the balloon, the art has provided strips of radiopaque markers on the catheter shaft, embedded radiopaque particles within the balloon wall, coated a portion of the interluminal balloon surface with radiopaque material, and flushed a radiopaque liquid through the balloon during inflation. The radiopaque material is then typically visualized by fluoroscopy. However, each of these prior art approaches poses difficulty in manufacturing and use of the balloon catheter systems that limit their usefulness or marketability.

For example, to guide a catheter through what is often a tortuous and diameter-compressed bodily lumen, it is key that the catheter be flexible. However, coating the catheter with radiopaque bands stiffens it at the application site, and often exposes the catheter material (usually a polymer) to melt temperatures that can cause warp of the catheter shaft.

Another critical parameter for an intraluminal catheter is its profile. The narrower the overall catheter system, the more flexible it is and the more susceptible it will be to use in a wider variety of vessel sizes. Yet embedding radiopaque particles within a balloon wall requires use of relatively thick balloon materials to enable a sufficient concentration of particles to be provided for visualization.

Coating the interior luminal surface of a balloon allows use of thinner balloon materials, but requires coating and finishing of the balloon prior to catheter mounting, limiting manufacturing options for the system. Further, if the balloon is not fully radiolucent (either because the balloon polymer isn't radiolucent, or because it is coated or wrapped with non-radiolucent reinforcements), visualization of the radiopaque material within the balloon can be impaired.

While one might be able to use such an intraluminally coated balloon without reinforcement, balloon resistance to breakage on overinflation is a critical concern, in that such breakage can have severe adverse effects on the patient. In certain prior art devices, reinforcements (such as non-compliant braids) have been applied to only portions of the outer surface of a balloon which has a radiopaque coating or is placed over a catheter with radiopaque bands, to allow visualization thereof or of a radiopaque fluid introduced into the balloon for inflation. Yet failing to provide reinforcements that are co-extensive with substantially the entire surface of the balloon provides the latter with inherent points of weakness, diminishing safety.

Accordingly, the art would significantly benefit from availability of a balloon catheter with improved radiopaque characteristics and a fully reinforced, break resistant compliant balloon.

### SUMMARY OF THE INVENTION

The present invention provides a compliant catheter balloon having improved wall integrity and radiopaque properties to facilitate accurate and safe intraluminal placement and inflation of the balloon within body cavities. In particular, the invention provides a fully radiopaque balloon with co-extensive reinforcement by non-compliant fibers, wherein the radiopaque balloon material is visualizable in an unobstructed manner within an intraluminal space. In preferred embodiments, the radiopaque material is disposed on the balloon in a fashion that aids in its folding. In especially preferred embodiments, the radiopaque coating is disposed on the balloon in a fashion which negates the need for use of any contrast media for visualization of the balloon during the procedure. In such embodiments, saline may be used as the sole inflation medium.

Accordingly, in one aspect, a radiopaque balloon for use with an intraluminal catheter is provided. The balloon includes an inner inflation layer, including a compliant polymeric cylinder defining a lumen for retention of inflation fluid. A fiber layer, is disposed on the inflation layer. The fiber layer includes at least two layers of inelastic, non-braided fibers disposed around the length of the inner wall by adhesive means, with the fibers of each layer separated by the adhesive means. Use of non-braided fibers improves inflation control by eliminating the potential for inter-fiber expansion.

In one embodiment the fiber layer includes a first layer of at least one inelastic, non-braided fiber helically disposed around the inner wall the fiber having a helical pitch extending along the longitudinal axis of the lumen. In another embodiment, the fiber layer includes (i) a first layer of at least one inelastic, non-braided fiber helically disposed around the inner wall and (ii) a second layer of at least one braided fiber disposed on the first layer around the length of the inner wall, with the fibers of each layer separated by adhesive means. In the various embodiments, the fiber layer is adhesively attached via impregnating the fiber layer with adhesive means after the fiber layer is disposed over the inflation layer. The pitch may be varied along the longitudinal axis extending along the length of the balloon to define regions having increased reinforcement.

In various embodiments, the balloon further includes a radiopaque material disposed over substantially the entire length of the balloon, preferably the entire length, in or on the fiber layer. In one embodiment, the adhesive means is a cured adhesive, and the radiopaque material is admixed with the adhesive prior to curing. In another embodiment, the radiopaque material is deposited onto the outermost surface of the fiber layer. In yet another embodiment, the radiopaque material is embedded in substantially all of the fibers of the fiber layer. A coating layer is disposed over the fiber layer including at least one layer of compliant radiolucent polymeric material.

In another aspect, radiopaque material is disposed over substantially the entire length of the balloon in the outer coating layer rather than in the fiber layer. Accordingly, the balloon includes an inner inflation layer, including a compliant polymeric cylinder defining a lumen for retention of inflation fluid. The balloon further includes a fiber layer, disposed on the inflation layer. The fiber layer includes at least two layers of inelastic, non-braided fibers disposed around the length of the inner wall by adhesive means, with the fibers of each layer separated by the adhesive means. The balloon further includes an outer coating layer including at least one layer of compliant polymeric material disposed around the fiber layer, the coating layer including radiopaque material disposed over substantially the entire length of the coating layer.

In another aspect, radiopaque material is disposed over substantially the entire length of the balloon, preferably the entire length, by applying a single layer of the material on the inflation layer rather than in the fiber layer or the coating layer. Accordingly, the balloon includes an inner inflation layer, including a compliant polymeric cylinder defining a lumen for retention of inflation fluid. The balloon further includes a fiber layer disposed on the inflation layer. The fiber layer includes at least two layers of inelastic, non-braided fibers disposed around the length of the inner wall by adhesive means, with the fibers of each layer separated by the adhesive means. The balloon further includes an outer coating layer including at least one layer of compliant radiolucent polymeric material.

In methods for use of the balloon of the invention to perform an intravascular surgical procedure, the balloon is mounted on an appropriate catheter and advanced through a body vessel of a subject to a treatment site. Where the balloon is coated along substantially its entire length with a radiopaque coating, and especially when substantially the entire surface of the balloon is covering by the coating, inflation is achieved using only saline as an inflation medium. Use of contrast media for visualization of the balloon during the procedure is avoided, and deflation times prior to removal of the balloon from the body are markedly increased; e.g., by at or around 50% compared to the time required for deflation of a balloon containing contrast medium.

### BRIEF DESCRIPTION OF THE DRAWINGS

FIG. 1 is a diagram showing an inflated catheter balloon having proximal (A) and distal (B) ends.

FIG. 2 is a diagram showing a lateral cross-section of one embodiment of a balloon in the inflated state.

FIG. 3 is a diagram showing an expanded cross-section of one embodiment of a balloon wall including an expanded view of the fiber layer 30.

FIG. 4 is a cross-sectional diagram of one embodiment of the balloon device showing the surface of the inflation layer 200 having a layer of radiopaque material 210 deposited thereon.

FIG. 5 is an illustration showing a portion of a braided fiber sheath utilized in one embodiment of the balloon device.

FIG. 6 is an illustration of one embodiment of the balloon device including a non-braided fiber helically disposed around the inner inflation layer with differing pitch along the length of the balloon.

FIG. 7 is an illustration showing the helical wrapping of a non-braided fiber disposed around the inner inflation layer in one embodiment of the balloon device.

### DETAILED DESCRIPTION OF THE INVENTION

The present invention is based on innovative designs for compliant radiopaque catheter balloons having increased radiopaque properties and wall integrity. The increased radiopaque properties improve accurate placement of the device within the stenosis and avoid the use of radiopaque inflation fluid; i.e., contrast media.

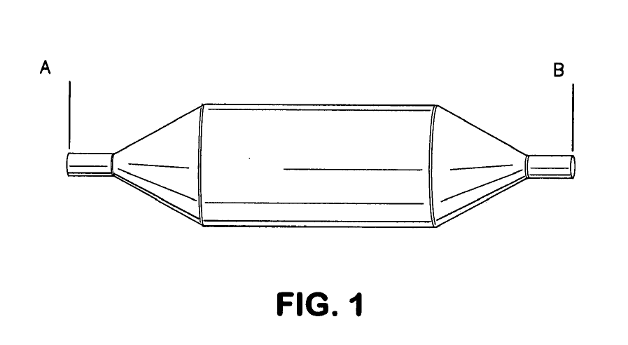

FIG. 1 generally shows the shape of a catheter balloon of the present invention. The balloon includes both distal (A) and proximal (B) ends with the longitudinal axis running from distal (A) and proximal (B) ends through a center lumen. At least the proximal (B) end may be configured for attachment over a portion of a catheter body. A variety of catheters are well known in the art and suitable for use with the balloon of the present invention.

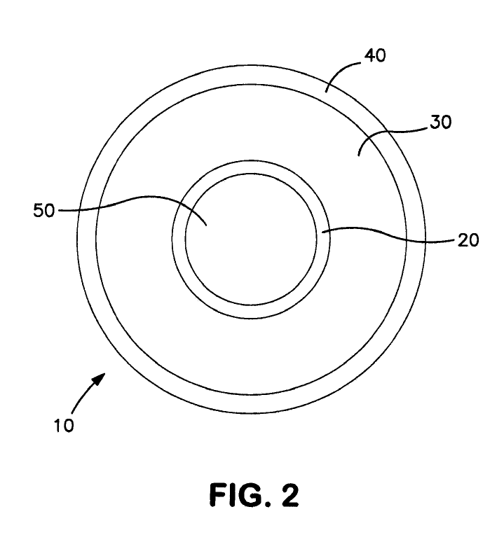

FIG. 2 generally shows a lateral cross-section across the width of one embodiment of an inflated balloon 10 of the present invention. The balloon 10 includes an inflation layer 20, a fiber layer 30, and a coating layer 40. The inflation layer 20 defines a lumen 50 for retention of inflation fluid used to increase the internal pressure of the lumen 50 to inflate the balloon 10. The lumen 50 is of sufficient diameter to accommodate a guidewire lumen allowing insertion of a guidewire therethrough and may be of variable diameter so as to attach to a variety of catheter types. The inflation layer 20 may be made of a compliant material which resiliently deforms under radial pressure. Examples of suitable compliant materials are generally known in the art and include materials such as, but not limited to polyethylene (PE), polyurethane (PU), nylon, silicone, low density polyethylene (LDPE), polyether block amides (PEBAX), and the like. In an exemplary embodiment, the inflation layer 20 is Vestamid® nylon.

The inflation layer 20 may be formed using any suitable method known in the art. For example, the inflation layer 20 may typically be blow-molded or formed on a mandrel to define the eventual shape of the inflated composite balloon 10. The balloon 10 is in a folded configuration in the deflated state, with folds running along the length of the balloon 10 from the distal (A) to proximal (B) ends. When inflated, the balloon 10 takes the shape of the inflation layer 20. Use of inelastic fibers disposed in layers or as a braided sheath disposed around the inflation layer 20 allows the original shape of the inflation layer 20 to be maintained through successive inflation and deflation cycles. Additionally, the original shape of the inflation layer 20 defines the shape of the fully assembled balloon 10 in the inflated state for use with a patient as the inelastic fibers maintain the integrity of the assembled balloon wall and substantially prevent radial distortion of the original blown shape when the balloon 10 is inflated within the stenosis of a patient. Further, utilizing inelastic fibers in the fiber layer 30, allows the wall thickness of the inflation layer 20 to be similar to those typically known in the art, or much thinner while continuing to avoid bursting or substantial radial distortion. Thus the wall thickness of the inflation layer 20 need only be thick enough to facilitate applying the fiber layer 30 on the inflation layer 20.

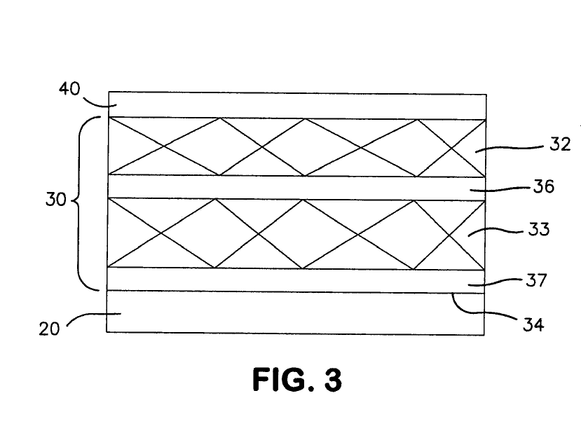

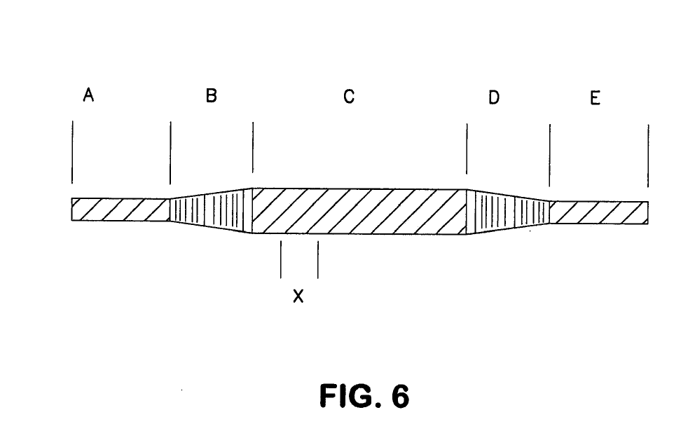

The fiber layer 30 is disposed on the inflation layer 20. In one embodiment, the layer is applied while the inflation layer 20 is in the expanded state. FIG. 3 shows an expanded cross-section of the balloon wall including an expanded view of the fiber layer 30. The fiber layer 30 may include one or more layers of inelastic fibers, for example, 32 and 33, disposed around the length of the inner wall 34 created by the outside surface of the inflation layer 20. Each layer of inelastic fiber may be separated by at least one layer of an adhesive means 36 used to apply the fibers. Typically, each inelastic fiber layer includes a single fiber applied by wrapping the fiber onto the balloon in a particular orientation to form the fiber layer. While the inflation layer 20 is in the inflated state, an adhesive means is applied to the wall 34 of the inflation layer 20. A single layer of inelastic fiber 33 is then applied to the surface. The “wrap” of the inelastic fiber may be of any suitable orientation that facilitates reinforcement of the inflation layer 20. For example, the fiber may be applied by wrapping the first inelastic fiber radially around the circumference of the surface of the inflation layer 20 along the length of the balloon from distal (A) to proximal (B) ends or parallel to the longitudinal axis of the balloon along its length from distal (A) to proximal (B) ends. Thus, in certain embodiments, the one or more fibers may be helically disposed around the inflation layer 20, the helix extending along the longitudinal axis running from distal tip (A) to proximal tip (B), and having a helical pitch, the circular helix being either right or left handed. As is known in the art, the helical pitch is the width of one complete helix turn, measured along the helix axis, as exemplified by distance X shown in FIG. 6.

One or more layers of an adhesive means may be applied over the first inelastic fiber layer 33 followed by wrapping of another inelastic fiber to create a second inelastic fiber layer 32 separated from the first inelastic fiber layer by one or more layers of the adhesive means 36. Layers of adhesive means may be allowed to cure or dry between each application of the adhesive means to impart additional thickness between successive inelastic fiber layers. Additional inelastic fiber layers may be applied in the same manner. Accordingly, fiber layer 30 may include 2, 3, 4, 5, 6, 7, 8, 9 or more individual inelastic fiber layers, each separated by one or more layers of an adhesive means.

Successive layers of inelastic fiber may be applied in any orientation with respect to the preceding inelastic fiber layer. For example, the second inelastic fiber 32 may be applied such that the fiber is perpendicular to the first fiber layer 33 or forms an angle from 90 (perpendicular) to 180 (parallel) degrees with respect to the wrap of the preceding inelastic fiber layer. In an exemplary aspect, the fiber of each successive inelastic fiber layer is applied perpendicular to the fiber of the preceding layer, with the first inelastic fiber layer 33 being applied radially around the circumference of the surface of the inflation layer 20 along the length of the balloon.

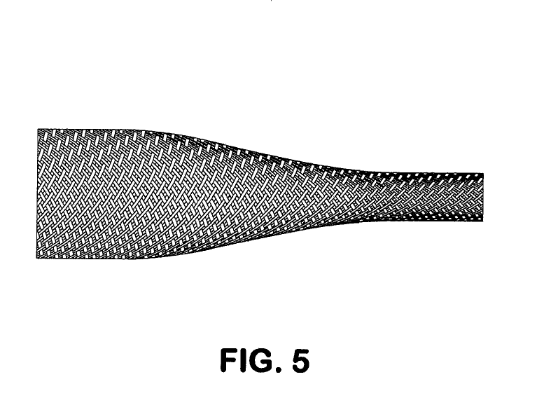

In an exemplary embodiment, fiber layer 30 includes a layer of inelastic fiber configured as a braided sleeve disposed over the inflation layer 20. As is well known in the art, a braid is typically a complex structure or pattern formed by intertwining two, three or more strands of flexible material such as textile fibers, wire, or the like. Inelastic fibers may be braided to form a hollow, generally cylindrical braided sleeve which may be disposed over inflation layer 20 and substantially prevent radial distortion of the original blown shape when the balloon 10 is inflated. A typical braided sleeve for use with the present invention is shown in FIG. 5 (showing the proximal or distal end of a braided sleeve).

As will be appreciated by one of skill in the art, various fiber configurations may be braided to form the sleeve. For example, individual fibers composed of an individual thread may be braided together as well as individual fibers composed of multiple threads, for example, individual threads braided to form a unitary braided fiber which is used to construct the braided sleeve. Thus the braided sleeve may be formed from inelastic fibers of any configuration, e.g., fibers of single or multiple threads, so long as the formed braided sheath prevents radial distortion of the balloon 10 when inflated. Accordingly, various embodiments fiber layer 30 may include 2, 3, 4, 5, 6, 7, 8, 9 or more inelastic fibers.

The braided fiber sleeve may be placed over inflation layer 20 by sliding the sleeve over the inflation layer 20 in an inflated state. The sleeve may then be pulled at distal and proximal ends to tighten the sleeve and affixed to inflation layer 20 at both proximal and distal ends by adhesive means. The sleeve may be optionally affixed to inflation layer 20 by adhesive means along the length of the balloon from proximal to distal ends in its entirety or any regions thereof. To obtain optimal burst pressures and maintain balloon size, e.g., both diameter and length during inflation, the braided fiber sleeve must be affixed to the inner balloon. As discussed herein, this may be accomplished by adhesive means as well as formation of coating layer 40.

In one embodiment, the fiber layer includes a first layer of non-braided fiber and a second layer of braided fiber or braided fiber sleeve. For example, one or more non-braided inelastic fibers may be helically applied along the length of the balloon before the braided fiber sleeve is disposed over the inflation layer. The fiber may have a different helical pitch or spacing in different regions of the balloon to provide regions with additional reinforcement. The first layer of fiber layer 30 may be formed by directly applying the fiber to the inflation layer 20 with or without adhesive. The fiber may be applied by applying a thin coat of adhesive to the outer surface of the inflation layer 20 and helically winding a non-braided inelastic fiber around the outer surface of the inflation layer 20 along the length of the balloon in various configurations such that the fiber layer 30 includes a first layer of non-braided fiber radially disposed around the outer surface of the inflation layer. Alternatively, the fiber may be dipped in adhesive prior to disposing the fiber on the inflation layer 20. As another alternative, the fiber is disposed around the inflation layer and the coating layer 40 is directly applied over the fiber layer 30. As another alternative, the fiber is disposed on the balloon along with an upper fiber braid and adhesive used to impregnate the fiber layer 30.

To provide additional burst resistance at specific regions along the balloon, the non-braided fiber may be applied at differing helical pitches along the length the balloon so that more or less fiber is deposited in specific regions. With reference to FIG. 6, the balloon of the present invention includes 5 discrete regions disposed along the longitudinal axis of the balloon including a distal tip region (A), a distal conical region (B), a central inflation region (C), a proximal conical region (D), and a proximal tip region (E). In various embodiments, at least one non-braided inelastic fiber may be helically wound such that more fiber is disposed on either, or both conical regions (B) and (D). The fiber may be wound with a high pitch in regions (A), (C) and (E), as compared to a low pitch for conical regions (B) and (D) to facilitate more fiber being deposited in regions (B) and (D). For example, in both or either regions (B) and (D), the fiber may be wound having virtually no pitch so that the fiber is essentially perpendicular to the longitudinal axis of the balloon (e.g., wound parallel to each other) and wound tightly so that the rings of fiber touch each other.

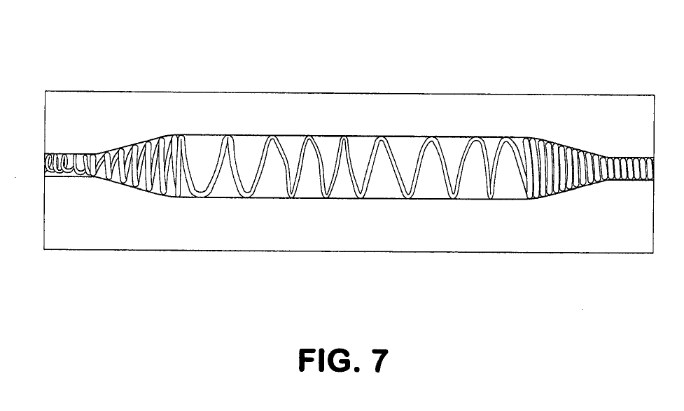

Inclusion of one or more non-braided inelastic fibers underneath the braided fiber sleeve allows one to impart additional burst characteristics. For example, greatly reinforcing the proximal end, e.g., regions (D) and/or (E), and not the distal end regions ensures that the balloon is more likely to burst at the distal end. This allows the physician to more easily remove the balloon from the vessel of a patient in the event the balloon ruptures during a procedure. Thus in various configurations, the non-braided inelastic fiber may be radially wound as shown in FIG. 7. The fiber is wound with a high pitch in regions (A), (B) and (C), with the pitch in region (C) being wider than that in regions (A) and (B), and virtually no pitch in regions (D) and (E) at the proximal end of the balloon.

One of skill in the art would appreciate the various combinations that are possible with regard to reinforcing regions along the length of the balloon using a non-braided fiber. With reference to FIG. 6, the pitch may be varied such that any of regions (A), (B), (C), (D) and/or (E) includes from about less than 0.1, 0.5, 1, 5 or 10 winds per mm to greater than about 50, 100, 250 or 500 winds per mm. Further, fibers disposed under the braided sheath may be of any thickness. However, in exemplary embodiments, the fibers will have a denier greater than or equal to about 25, 30, 35, 40, 45, 50, 75, 100, 500, 1000, 1500, 2000 or 2500 denier.

In various embodiments, the helical pitch may remain constant or vary for a specific discrete region to define specific bursting characteristics. In one embodiment, the helical pitch in either or both the proximal conical region (D) and/or distal conical region (B) is at least 10, 20, 30, 40, 50, 60, 70, 80, 90, 100, or 1000 times less than the pitch in the distal tip region (A), the proximal tip region (E), and/or the central region (C). In another embodiment, the helical pitch in the proximal conical region (D) and the proximal tip region (E) is at least 10, 20, 30, 40, 50, 60, 70, 80, 90, 100, or 1000 times less than the pitch in the other regions. The helical pitch in any combination of discrete regions may be at least 10, 20, 30, 40, 50, 60, 70, 80, 90, 100, or 1000 times less than the pitch in any of the remaining regions.

Along with depositing the one or more non-braided fibers by helically winding the fiber around inflation layer 20, it will be appreciated that the fibers may be pre-formed and disposed over inflation layer 20 in a manner similar to placing the braided fiber sleeve over the inflation layer 20. For example, a pre-form may be constructed of one or more regions (A), (B), (C), (D) and/or (E), which may be assembled on to inflation layer 20 before the braided sleeve is slid over the inflation layer 20. The pre-form may be designed to fit over only a single regions, e.g., conical regions (B) or (D) of the balloon or may be configured to simultaneously fit one or more additional regions of the balloon, e.g., regions (A), (C) and (E). In various embodiments the pre-form is configured to be disposed over only either or both conical regions (B) and/or (D), regions (A) and/or (E), or over all regions of the balloon.

As used herein, “adhesive means” includes any suitable adhesive, glue, manufacturing process, such as thermobonding, or combination thereof, known by one of skill in the art that may be used for attaching successive layers of inelastic fibers.

The fiber utilized in the inelastic fiber layer(s) and/or sleeve may be a braided or non-braided fiber. As used herein, non-braided means that the fiber is not intertwined to form a three-dimensional structure. The inelastic fibers are of high-strength and typically made of a high-strength polymeric material. Examples of suitable materials are generally known in the art and include materials such as, but not limited to Kevlar®, Vectran®, Spectra®, Dacron®, Dyneema®, Terlon® (PBT), Zylon® (PBO), polyimides (PIM), other ultra high molecular weight polyethylene (UHMWPE), aramids, and the like. The inelastic fibers are characterized by high tensile strength and have minimal elasticity or stretch. For example, Kevlar® is a spun fiber having a high tensile yield strength of about 3,620 Mpa with a relative density of about 1.44 as compared to an elastic nylon fiber typically has a tensile yield strength of less than about 50 Mpa with a relative density of about 1.15. Accordingly, in an exemplary embodiment, inelastic fibers for use with the present invention have a high tensile yield strength of greater than about 2,000, 2,500, 3,000, 3,500 Mpa or higher.

In various embodiments, a coating layer 40 is disposed around the fiber layer 30. The coating layer 40 is composed of one or more layers of a compliant polymeric material. One or more layers of the compliant polymeric material of the coating layer 40 may be composed of the same material used to form the inflation layer 20. Alternatively, coating layer 40 may be of a different material than that used to for the inflation layer 20. Examples of suitable materials are generally known in the art and include materials such as, but not limited to polyethylene (PE), polyurethane (PU), nylon, silicone (e.g., silicone sealants and adhesives), low density polyethylene (LDPE), polyether block amides (PEBAX), and the like. In an exemplary embodiment, coating layer 40 comprises a UV/visible light curable silicone coating of low durometer, such as Loctite® 5055. In an exemplary embodiment, inflation layer 20 and coating layer 40 are composed of different materials, inflation layer 20 being composed of a nylon (e.g., Vestamid® nylon) and coating layer 40 being composed of a silicone (e.g., Loctite® 5055).

The coating layer 40 may be applied in any number of ways as are known in the art, for example, as either a liquid or spray coating. Typical coating methods include spray coating, dip coating, dispense coating, pad printing and the like. One or more layers of material may be successively applied in spray or liquid form around the fiber layer 30 until a suitable thickness of the coating layer 40 is obtained, optionally allowing the material to dry or cure between applications, with the same or different coating materials being applied each application.

As discussed herein, to obtain optimal burst pressures and maintain balloon size, e.g., both diameter and length during inflation, the braided fiber sleeve must be affixed to the inner balloon. This may be accomplished by application of coating layer 40. The materials used to form coating layer 40 exhibit adhesive characteristics which allows the material used for the coating layer to adhesively affix the braided fiber sleeve to the inflation layer 20 along the length of the balloon from proximal to distal ends in its entirety by penetrating the braided fiber sleeve and acting to adhere the sleeve to inflation layer 20 while simultaneously forming outer coating layer 40. In an exemplary aspect, coating layer 40 is formed of silicone (e.g., Loctite® 5055) which allows for adhesion of the braided fiber sleeve to inflation layer 20 such that upon inflation of the balloon, the diameter of the balloon is increased to a fixed diameter while the length experiences substantially no change.

The present invention provides balloons in which the integrity of the assembled balloon wall is preserved by inclusion of fiber layer 30 which substantially prevents radial distortion of the original blown shape of the inflation layer 20 when balloon 10 is inflated. Balloon 10 exhibits the flexibility and elastic characteristics of an elastomeric material, but also has a well-defined growth limit such as is exhibited by inelastic balloons to prevent over inflation and bursting of the balloon within the blood vessel of the patient to prevent rupture of the stenosis. To accommodate various sized blood vessels, balloons of the present invention may be sized to have well defined maximum diameters when inflated. For example, balloons may have a maximum inflation diameter of from 5 to 20 mm. Additionally, balloons of the present invention have a relatively high rated burst pressure of greater than 20 atmospheres, e.g., greater than 20, 21, 22, 23, 24, 25, 26, 27, 28, 29, or 30 atmospheres. Typically the balloons have a rated burst pressure of between 20 and 30 atmospheres.

As one of skill in the art would appreciate, balloon of different maximum inflation diameters may exhibit different burst pressures. It is contemplated that balloons having a maximum inflation diameter of from 5 to 10 mm exhibit a rated burst pressure of between 25 to 30 atmospheres, while balloons having a maximum inflation diameter of from 12 to 20 mm exhibit a rated burst pressure of between 16 to 22 atmospheres. In view of the fiber reinforcement to balloons of the invention, however, resistance to rupture at relatively high pressures (e.g., on overinflation) compared to unreinforced balloons is provided.

When deflated, the fully constructed balloon of the present invention is typically folded with pleats extending longitudinally along the length of the balloon and defined by a minimum balloon diameter (dₘᵢₙ). Upon inflation, the fully constructed balloon expands to a defined maximum inflated diameter (dₘₐₓ). The dₘᵢₙ of the balloon ranges from a dₘᵢₙ of approximately 1.6 to 2.6 mm, while the dₘₐₓ ranges from approximately 5 mm to a dₘₐₓ of approximately 20 mm.

In various embodiments, the balloon further includes a radiopaque material disposed over substantially the entire length of the balloon (i.e., along substantially all of its surface area). The radiopaque material may be included in one or more of the various balloon layers such that the radiopaque material is disposed over substantially the entire length of the balloon from proximal tip to distal tip. Alternatively, the radiopaque material may be deposited entirely over the ‘working’ length of the balloon, e.g., region (C) of FIG. 6, while being excluded from distal regions (A) and (B) and distal regions (D) and (E) regions.

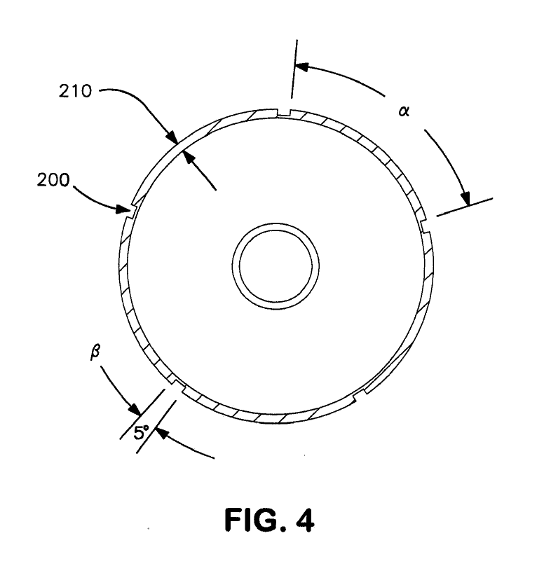

In various embodiments, the radiopaque material may be disposed in any pattern over the balloon. For example, as shown in FIG. 1 and the cross-section shown in FIG. 4, the radiopaque material may form a longitudinal striped pattern over the entire length of the balloon from the proximal end to the distal end, over the ‘working’ length of the balloon, e.g., region (C), or over any portion of the balloon. Similarly, the radiopaque material may be disposed over the full radius of the balloon over the entire length of the balloon from proximal to distal ends as shown in FIG. 1, over the ‘working’ length of the balloon, or over any portion of the balloon, e.g, forming any number of bands along the length of the balloon. In one embodiment the radiopaque material may be disposed as radial bands spaced along the entire length of the balloon or any portion thereof, for example, one or multiple bands at one or each of the proximal and distal tips. In an exemplary embodiment, radiopaque material is disposed over the ‘working’ length of the balloon, and at the distal tip region (A), or disposed over substantially the full length of the balloon including regions (A) to (E).

In various embodiments the radiopaque material is included within the fiber layer 30. For example, the adhesive means may be a cured adhesive, and the radiopaque material is admixed with the adhesive prior to curing. Alternatively, the radiopaque material may be applied directly to the adhesive after it is applied to the balloon. As such, the radiopaque material may be applied via the adhesive means such that it is disposed in one or more adhesive layers of the fiber layer 30.

In another embodiment, the radiopaque material is deposited onto the outermost surface of the fiber layer 30. The outermost surface of the fiber layer 30 may be one or more layers of adhesive means, or the outermost layer may be an inelastic fiber layer included within the fiber layer 30, or a combination thereof.

In another embodiment, the radiopaque material is embedded in one or more of the inelastic fibers composing the inelastic fiber layers of the fiber layer 30. For example, the radiopaque material may be added to the inelastic fiber material before the fibers are spun or extruded. The radiopaque material may be included in any number of the inelastic fiber layers. For example, the radiopaque material may be included in one to substantially all of the inelastic fibers of the fiber layer.

In another embodiment, the radiopaque material may be included in one or more layers of the compliant polymeric materials included in the coating layer 40 disposed around the fiber layer 30. For example, the radiopaque material may be admixed with the compliant polymeric material before it is applied to the fiber layer 30. Alternatively, the radiopaque material may be applied directly to compliant polymeric material after it is applied to the balloon.

In another embodiment, the radiopaque material may be applied directly to the wall 34 of the inflation layer 20. As such, the radiopaque material may be disposed over substantially the entire length of the balloon by applying a single layer of the material on the inflation layer 20 rather than in the fiber layer 30 or the coating layer 40. The radiopaque material may be applied in several applications as discussed further herein, to achieve the desired radiopacity of the single layer. Alternatively the radiopaque material may be applied to discrete regions of the wall 34 of the inflation layer 20 in any pattern.

FIG. 4 shows an embodiment in which the radiopaque material 210 is deposited directly on the outer surface of the inner layer 200. As one of skill in the art would appreciate, in various embodiments where the radiopaque material forms a striped pattern, angle α may range from 0 degrees (e.g., no radiopaque material) to 360 degrees (e.g., a continuous annular coating of radiopaque material) to define virtually any stripe pattern. Likewise, angle β may range from 0 degrees (e.g., no radiopaque material) to 360 degrees (e.g., a continuous annular coating of radiopaque material) to define virtually any stripe pattern. Thus any combination of α or β may be used and each may be about 0-5, 5-10, 10-15, 15-20, 20-25, 25-30, 30-35, 35-40, 40-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-155, 155-160, 160-165, 165-170, 170-175, 175-180, 180-185, 185-190, 190-195, 195-200, 205-210, 210-215, 215-220, 220-225, 225-230, 230-235, 235-240, 240-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-355 or 355-360 degrees.

As discussed herein, the longitudinal stripes may extend over the ‘working’ length of the balloon, or over substantially the full length of the balloon including regions (A) to (E). In various embodiments, the total number of stripes extending longitudinally around the radius of the balloon may be 0, 1, 2, 3, 4, 5, 6, 7, 8, 9, 10, 11 or more, which may be equally spaced around the circumference of the inner layer 200. In an exemplary embodiment, 5 stripes are provided with α equal to 67 degrees and β equal to 5 degrees as shown in FIG. 4.

With regard depositing the radiopaque material 210 directly on the outer surface of the inner layer 200, it has been determined that such configuration assists with folding of the balloon upon deflation. As shown in FIG. 4, spacing provided between the longitudinal stripes allows the folded balloon to conform to have a reduced inflated diameter which assists in inserting or removing the device in a patient's vessel. In the deflated state, a balloon may have 1, 2, 3, 4, 5, 6, 7, 8, 9, 10, 11, 12, 13, 14, 15 or more folds.

In various embodiments, the radiopaque material may be applied at varying thicknesses. In one embodiment where the radiopaque material is deposited directly in the outer surface of the inflation layer 20, the material is deposited at a thickness of less than about 0.004 inches, most preferably less than 0.001 inches. For example, the radiopaque material may be deposited per side of the inflation lumen or any other balloon embodiment or element at a thickness of about 0.0001-0.0005, 0.0005-0.0007, or 0.0005-0.0009 inches.

In embodiments where the radiopaque material is applied to layers underlying the coating layer, the coating layer may be comprised of one or more layers of a radiolucent polymeric material, preferably a compliant polymeric material. The radiolucent polymeric material ensures that visualization of radiopaque material in any of the underlying layers is not obstructed.

Use of the radiopaque material in various layers of the balloon allows the balloon to be constructed with control over the desired radiopaque properties of the finished balloon. For example, a balloon may be constructed including radiopaque material along substantially the entire length of the balloon in which the amount of radiopaque material may be increased or decreased with ease depending on the type, number, thickness and disposition of the layers.

A variety of radiopaque materials are well known and suitable for use with the present invention. Such materials include, but are not limited to barium, bismuth, tungsten, iridium, iodine, gold, iron, and platinum. A single radiopaque material may be used or such materials maybe mixed in various ratios to provide the desired radiopacity. As will be appreciated by one of skill in the art, different radiopaque materials may disposed on/in different regions of the balloon in various combinations the achieve the desired radiopacity. For example, one radiopaque material or combination thereof may be used at the distal tip while a different radiopaque material or combination thereof may be used along the length of the balloon extending from the distal tip (B) to the proximal tip (A). In an exemplary embodiment, the radiopaque material is entirely or predominantly tungsten. For example, balloon components, such as fibers, inks, adhesives and/or polymeric materials may be loaded with tungsten at greater than 90, 91, 92, 93, 94, 95, 96, 97, 98 or 99 percent. Exemplary inks may include epoxy or urethane based inks loaded with greater than 90, 95 or 99 percent tungsten. Exemplary adhesives and/or polymeric materials include polyurethane or polyimide loaded with greater than 90, 95 or 99 percent tungsten.

As discussed herein, the radiopaque material may be incorporated into various layers through admixing the material with, for example, the adhesive, polymeric coating material, or inelastic fiber material. However, the radiopaque material may also be applied by any other method known in the art. Such methods include, but are not limited to coatings, electroplating, chemical vapor deposition (CVD), physical vapor deposition (PVD), and ion beam assisted deposition (IBAD). One or more methods may be employed depending on the desired characteristics of the radiopaque layer, such as thickness, flexibility, radiopacity and the like. Additionally, one layer of radiopaque material may be directly applied to the surface of another. Typically, the radiopaque materials will be admixed with inks, adhesives and/or polymeric coating materials and coated onto one or more layers of the balloon 10. As such, the radiopaque material may be coated onto a layer of the balloon by spray coating, dip coating, dispense coating, printing, or the like.

The present invention further provides innovative balloon configurations that allow for increased inflation and deflation performance utilizing preferred inflation fluids, such as unmixed saline solution or solutions wherein the saline component is 70% or greater. For example, the present balloon is capable of utilizing only saline solution, or mixtures of saline and contrast media where the saline component is present at 70, 75, 80, 85, 90, 95, 99 percent or greater. The balloon design accommodates inflation fluids having a high saline solution content and exhibits a faster rate of deflation as compared to a conventional balloon that utilizes a mixture of inflation fluids, wherein the ratio of saline solution to contrast media is less than 70:30. Conventional balloons typically require use of inflation fluids including a mixture of saline solution and contrast media, wherein the fluid includes at least 50% or more of the contrast media component. As compared with such conventional balloons, the balloons of the present invention exhibit faster deflation rates of at least 10, 15, 20, 25, 30, 35, 40, 45, 50, 55 or 60% greater, typically at least 50% greater, as compared with conventional balloons utilizing contrast media alone or mixtures of inflation fluids having a low saline solution content, e.g., 60-50% or less.

As such, the invention also provides a method of performing a surgical procedure using a catheter including the balloon device of the present invention, wherein the balloon exhibits increased deflation rates as compared with a conventional balloon. The method includes introducing a catheter having a balloon of the present invention into the vessel of subject. Inflating the balloon by introducing pressurized fluid into the inflation layer of the balloon, wherein the fluid consists of saline. Then deflating the balloon by decreasing the pressure of the fluid within the inflation layer of the balloon, wherein the balloon deflates at an increased rate as compared with a convention balloon, and withdrawing the balloon from the vessel of the patient.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Fabrication of Compliant Radiopaque Balloon Having a Braided Fiber Layer

Balloons were constructed having the general cross-sectional design configuration depicted in FIG. 2. Shown in an inflated state, the balloons generally included an inflation layer 20, a fiber layer 30, and a coating layer 40. The inflation layer 20 defines a lumen 50 for retention of inflation fluid used to increase the internal pressure of the lumen 50 to inflate the balloon 10. With reference to FIGS. 2 and 4, the balloons included the following components: inner balloon or inflation layer 20, fiber layer 30, coating layer 40, and deposited directly on the outer surface of the inflation layer 200 is radiopaque layer 210. Materials used for each component are shown in Table 1 as follows.

TABLE 1
Balloon Component Materials List

| Balloon Component | Material Description/Specification |
|---|---|
| Inflation Layer | Vestamid ® Nylon |
| (Inner Balloon) (20) | |
| Radiopaque | Epoxy based ink with >95% tungsten |
| Coating (210) | |
| Fiber Layer (30) | Ultra High Molecular Weight Polyethylene |
| | (UHMWPE) Fiber |
| Coating Layer | Loctite ® 5055 (nylon based polymer) |
| (Outer Polymer | |
| Coating/Adhesive) (40) | |

To construct the balloons, inflation layer 20 was first formed from compliant nylon material using a blow molding process. Next, radiopaque coating 210 was applied to the outer surface of the inflation layer 200 via printing in a longitudinally striped pattern along the ‘working’ length of the balloon (e.g., the region between the conical regions of the balloon) or to substantially the entire outer surface of the inflation layer 200. Fiber layer 30 was next formed by sliding a prefabricated braided fiber sleeve over inflation layer 20 and adhesively gluing distal and proximal ends of the sleeve to hold the sleeve in place. The fiber sleeve of fiber layer 30 was then impregnated with an adhesive polymer (Loctite® 5055) applied by spray coating to bond the individual fibers of fiber layer 30 to the substrate layer and form coating layer 40. Coating layer 40 was allowed to cure before assembly onto a catheter shaft.

Balloons having various maximum balloon inflation diameters were fabricated using the above described method for compatibility with catheters of 6, 7 or 8 French, although those of skill in the art will recognize that compatibility with other French sizes can be obtained through appropriate modifications of the balloon dimensions. The balloons have a rated burst pressure of 25-30 atmospheres for 5-10 mm diameter balloons and 16-22 atmospheres for 12-20 mm diameter balloons.

The balloons were then assembled upon an appropriately configured catheter. Typically, the balloons are assembled onto a catheter including a shaft having a distal tip which includes radiopaque material. The tip typically is composed of a Pebax material loaded with 20-40% radiopaque material, such as barium sulfate, bismuth and/or tungsten, prior to extrusion.

### EXAMPLE 2

### Fabrication of Compliant Radiopaque Balloon Having a Fiber Layer Including Braided and Non-Braided Fiber

Balloons were constructed in a process similar to that discussed in Example 1 with variations to the fiber layer 30. For example, a balloon was constructed including in which the fiber layer 30 includes both a first non-braided layer and a second braided fiber layer. The balloon materials are those shown in Table 1.

To construct the balloons, inflation layer 20 was first formed from compliant nylon material using a blow molding process. Next, radiopaque coating 210 was optionally applied to the outer surface of the inflation layer 200 via printing in a longitudinally striped pattern along the ‘working’ length of the balloon (e.g., the region between the conical regions of the balloon) or to substantially the entire outer surface of the inflation layer 200. Fiber layer 30 was next formed by applying a thin coat of adhesive to the outer surface of the inflation layer 200 and radially winding a non-braided inelastic fiber around the outer surface of the inflation layer 200 along the length of the balloon in various configurations such that the fiber layer 30 includes a layer of non-braided fiber radially disposed around the outer surface of the inflation layer.

In one configuration, the non-braided inelastic fiber was radially wound as shown in FIG. 6. The fiber was wound with a wide pitch in regions (A), (C) and (E), and with a narrow pitch in conical regions (B) and (D). In regions (B) and (D), the fiber is wound having virtually no pitch so that the fiber is essentially perpendicular to the longitudinal axis of the balloon and wound tightly so that the rings of fiber touch each other.

In another configuration, the non-braided inelastic fiber was radially wound as shown in FIG. 7. The fiber was wound in a wide pitch in regions (A), (B) and (C), with the pitch in region (C) being wider than that in regions (A) and (B), and virtually no pitch in regions (D) and (E) at the proximal end of the balloon.

After the non-braided fiber is applied, the adhesive was allowed to cure and a prefabricated braided inelastic fiber sleeve was applied as in Example 1. The prefabricated braided fiber sleeve was slid over inflation layer 20 having the non-braided fiber disposed thereon, and pulling the distal and proximal ends of the sleeve to tighten the sleeve over the balloon. The sleeve was then optionally adhesively glued at the distal and proximal ends before spray coating with additional adhesive polymer (Loctite® 5055) to bond the individual fibers of fiber layer 30 to the substrate layer and form coating layer 40. The adhesive is then allowed to cure to form coating layer 40 before assembly onto a catheter shaft.

### EXAMPLE 3

### Inflation and Deflation Rates of Balloons Utilizing Various Mixtures of Inflation Fluid

Inflation and deflation rates were tested for a balloon utilizing various ratios of saline to contrast media as inflation fluid. To perform the experiment, a 6 mm diameter by 10 cm balloon (Bard Dorado®) was tested. It is important to note that unlike the balloon of the present invention, the balloon used to perform the experiment requires the inflation fluid to include 50% or greater of contrast media in a surgical setting to be functional for the surgical procedure. Three trails were performed using saline alone and a 50:50 saline to contrast media mixture. The results are shown in Table 2 below.

TABLE 2
Balloon Catheter Inflation and Deflation Rates

| Balloon | | | | |
|---|---|---|---|---|
| Catheter | Saline | | 50/50 Contrast Saline | |
| Trials | Inflate (Sec.) | Deflate (Sec.) | Inflate (Sec.) | Deflate (Sec.) |
| 1 | 18* | 8 | 13 | 17 |
| 2 | 15 | 9 | 12 | 19 |
| 3 | 14 | 10 | 12 | 20 |
| Avg. | 14.50 | 9.00 | 12.33 | 18.67 |

As shown in Table 2, the deflation times observed show that increasing the viscosity of the inflation fluid by increasing the amount of contrast media to the saline/contrast media mixture, approximately doubles the amount of time required to deflate the balloon. Thus, the balloons of the present invention, capable of utilizing inflation fluids including a saline component of 70% or greater, exhibit deflation rates that are up to 50% faster as compared to conventional balloons requiring at least 50% contrast media in the inflation fluid for functionality.

Although the invention has been described, it will be understood that modifications and variations are encompassed within the spirit and scope of the invention. Accordingly, the invention is limited only by the following claims.

What is claimed is:

1. A radiopaque balloon for use with an intraluminal catheter, comprising:
(a) an inflation layer, consisting of a compliant polymeric cylinder having a central region ending in opposing conical regions, said cylinder defining an outer surface and an inner lumen for retention of inflation fluid;
(b) a fiber layer, consisting of at least two layers of inelastic, braided or non-braided fibers disposed around the length of the inflation layer;
(c) at least one coating layer, consisting of at least one adhesive disposed to affix the fiber layer to the inflation layer, wherein at least one adhesive penetrates the fiber layer to form the at least one coating layer; and,
(d) a radiopaque material disposed over substantially the entire length of the outer surface of the inflation layer.

2. The radiopaque balloon of claim 1, wherein the radiopaque material is deposited onto the outer surface of the inflation layer.

3. The radiopaque balloon of claim 1, wherein the radiopaque material is selected from the group of materials consisting of powdered tungsten, gold, iridium, platinum, barium, bismuth, iodine or iron.

4. The radiopaque balloon of claim 1, wherein each layer of the fiber layer is separated by the adhesive.

5. The radiopaque balloon of claim 1, wherein the fiber layer is disposed around the length of the inflation layer as a braided fiber sleeve.

6. The radiopaque balloon of claim 1, wherein the radiopaque material is disposed in a striped pattern along the length of the inflation layer.

7. The radiopaque balloon of claim 6, wherein the striped pattern comprises 1 to 15 stripes.

8. The radiopaque balloon of claim 7, wherein the striped pattern comprises 5 stripes.

9. The radiopaque balloon of claim 1, wherein the radiopaque material is deposited with a thickness of about 0.0001 to about 0.002 inches.

10. The radiopaque balloon of claim 1, wherein the radiopaque material is deposited with a thickness of about 0.0005 to about 0.0009 inches.

11. The radiopaque balloon of claim 1, wherein the lumen is of sufficient diameter to accommodate insertion of a guidewire therethrough.

12. The radiopaque balloon of claim 1, wherein at least the proximal tip is disposed over a portion of a catheter body.

13. The radiopaque balloon of claim 1, wherein the balloon has a rated burst pressure of between 16 and 22 atmospheres.

14. The radiopaque balloon of claim 1, wherein the braided fibers have a pitch with respect to one another between 65° and 75° along the central region of the balloon and a pitch with respect to one another between 35° and 45° at both conical regions of the balloon to provide the balloon with a burst pressure of 16 atmospheres or greater.
